(19) United States
(12) Patent Application Publication
(10) Pub. No.: US 2021/0241649 A1
(43) Pub. Date: Aug. 5, 2021

(54) SYSTEM AND METHOD FOR GUIDING A USER TO IMPROVE GENERAL PERFORMANCE

(71) Applicant: OURA HEALTH OY, Oulu (FI)

(72) Inventor: Hannu KINNUNEN, Oulu (FI)

(73) Assignee: OURA HEALTH OY, Oulu (FI)

(21) Appl. No.: 17/052,001

(22) PCT Filed: Jun. 18, 2019
(86) PCT No.: PCT/FI2019/050466

§ 371 Date: Oct. 30, 2020

(30) Foreign Application Priority Data
Jun. 28, 2018 (FI) 20185580

(51) Int. Cl. G09B 19/00; A61B 5/00; A61B 5/0205; G16H 20/30; G16H 40/67

(52) U.S. Cl. CPC G09B 19/00; A61B 5/4857; A61B 5/02438; G16H 20/30; G16H 40/67; A61B 5/02055

(57) ABSTRACT

A method and system for guiding a user to improve general performance. The system includes a wearable device to measure at least two parameters associated with the user during an activity period, and a computing device operatively coupled to the wearable device, wherein the computing device is operable to: determine a circadian rhythm of the user based on the measured parameters, classify the user into a chronotype class based on the determined circadian rhythm, determine a typical activity schedule for the user based on the measured parameters, determine an optimal time period in a typical day of the user for sleep and at least one of physical action and cognitive action, receive information about an intended activity of the user, analyse whether the intended activity is within the optimal time period, and guide the user on carrying out the intended activity.

### TECHNICAL FIELD

The present disclosure relates generally to analysis and processing of data related to circadian rhythm of a user; and specially to a system and method for providing guidance for improving general performance of a user.

### BACKGROUND

Typically, day and night rhythm of human beings is paced by a sleep duration, morning and evening activities (working hours, exercises and so forth), eating habits (breakfast, lunch and dinner) and so forth. There are variations in the way the day and night rhythm of a human being is aligned to a 24-hour circadian rhythm. That is, human beings' internal clocks are not identical, as each human being's biological or internal clock operates in their own natural rhythm (different from a 24-hour rhythm). For example, the body clocks of some human beings have a preference to operate with a slightly faster-paced rhythm than the exact 24-hour cycle, and others have internal clocks that are a bit slower than the 24-hour cycle. Moreover, everyone's day and night rhythm, also known as circadian rhythm, may be differently aligned with the 24-hour clock rhythm. In real life however, everybody needs to adopt to 24-hour sleep-wake cycle.

The circadian rhythms, user's sleep-wake cycle and daily activities (i.e. day and night rhythms related to 24-h clock rhythm) can be used to a classify persons to different chronotype classes or groups. For example, the slightly faster-paced individuals (whose natural day would have a length of for example 23 hours and 45 minutes) are often referred to as “morning people”, brisk, attentive and ready to go at dawn; whereas the slower-paced individuals (whose natural day would have a length of for example 24 hours and 20 minutes) are often referred to as “evening people”, reaching their peak performance at dusk. As there are people with different heights and weights, there are also people with different chronotype. According to research, chronotype seems to be much about genetics. While circadian alignment can change a bit according to age, it is however overall something that a person is born with.

Generally, each individual (coming under any one of the chronotypes) has a different pace and efficiency at different time periods, in between the 24-hour cycle of a typical day. Therefore, regular schedules of individuals vary depending on daily activities, eating habits, geographical locations, age group, genetics and so forth. Natural circadian rhythm gives a natural schedule for different activities and task to do with optimal efforts and at right timings. It is also natural that a human being is flexible to vary tasks and timing in certain range in short term. However, the performance may not be optimal or recovery may take long time, to return to a normal and typical schedule. During daily life a person may need to adapt his/her natural (chronotype depended or related) schedule of activities due to different reasons, for example due to temporal work-related task, such as a late-night phone call or attending sport competition etc. Currently there is no tool and method to do that effectively and guide the person adapt effectively to this new task and activity with updated schedule. However, difference in alignment of the circadian rhythms has a great impact on health and wellness of human beings. Furthermore, the difference in chronotype also creates a gap in coordination among the individuals (or a group of individuals), such as a decrease in average working hours, lower overlapping of an average working shifts among the individuals and so forth.

Additionally, due to irregularity of the circadian rhythm, the rate of success decreases abruptly for short term goals (plans to be fulfilled in days, weeks or in a month) and steadily for long term goals (plans to be fulfilled in months or in a couple of years). Moreover, regular tasks that are to be performed within a specific time period by an individual are disrupted by the uneven circadian rhythm. In order to maintain a healthy lifestyle, avoid stress and optimize resources in a balanced way, human beings should be aware of their own circadian rhythm and should regularise the activities accordingly.

Conventionally, systems and applications have been developed to help human beings in regularising and maintaining 24-hour circadian rhythm thereof. Such systems operate on user's input (such as height, weight, age and so forth) and present results as a regular program (schedule) accordingly. However, such results only present repeated programs for longer terms and do not improvise based on the circadian rhythm of the user. Additionally, such systems and applications have their limitations, such as non-optimized schedules, stagnant programs for long term, irregular back-ups and so forth.

Document US 2017/132,946 discloses a system for providing feedback to a user for improving performance level management. The system includes a wearable electronic device with means for measuring circadian rhythm and duration of sleep; a mobile communication device configured to communicate with the wearable electronic device; and a server configured to communicate with the mobile communication device, and operable to collect a first set of information from the user, calibrate the first set of information based on a set of measurement data from the wearable electronic device, set a target level of performance of the user, compare the measured circadian rhythm, the measured duration of sleep and time information against each other and a set of rules to determine the performance level of the user, compare the determined performance level to the target level of performance, and provide an alert and feedback when the determined performance level is below the target level of performance. Document US 2009/105,560 discloses a computerized system for scheduling at least one daily activity of a user. One or more sensors are attached to the body of the user which monitor one or more physiological parameters of the body Physiological data is produced representative of the one or more physiological parameters during a time period. A processing unit attached to memory, is programmed for the scheduling of activities based on the physiological data and on previously stored values. The scheduled activities preferably include eating of a meal, exercise or rest of the user. Physiological parameters include skin temperature and/or heart rate. When the scheduled daily activity is eating of a meal, the processing unit is preferably programmed to recommend to the user to eat the meal during a portion of the time period when the skin temperature is rising or when the heart rate is falling. Document US 2011/144,528 presents a personal health system which includes a suitable Core Body Temperature (CBT) monitor that can be worn for all or part of a 24 hour day and collect continuous CBT data. The CBT data is collected and compared to determine circadian desynchrony. A conveniently carried or worn processor/display unit, in communication with the CBT monitor, algorithmically determines activity types and activity timing based on the collected CBT data to improve synchrony. The activities and when to perform them are displayed to the user. Document US 2014/276,244 discloses a method for a time management system. The method receives inputs from a user where the inputs are for an activity associated with the user. An indication of the activity is displayed on a wearable device at a predetermined time. This displaying is predominate during a normal mode of operation. The method is performed by a software application where a primary focus of the software application is for time management for the user. Document U.S. Pat. No. 9,579,060 concerns a hat, helmet, and other headgear apparatus including dry electrophysiological electrodes and, optionally, other physiological and/or environmental sensors to measure signals such as ECG from the head of a subject. Methods of use of such apparatus to provide fitness, health, or other measured or derived, estimated, or predicted metrics are also disclosed. Document US 2002/019,586 presents a detecting, monitoring and reporting apparatus including at least two sensors for facilitating the generation of data indicative of physiological parameters of the individual and/or data indicative of a contextual parameters of the individual. A processor is coupled to the sensors and is adapted to generate at least one of derived data from at least a portion of the data indicative of physiological parameters and analytical status data from at least a portion of at least one of the data indicative of physiological parameters, the data indicative of contextual parameters, the derived data and the analytical status data. A memory retrievably stores the data and one of various ways of transmitting the data is provided.

The problem is that the user does not know what is a typical schedule for activities to follow. When the user has an intended activity (for example exercise, or cognitive work) for the day, the user cannot check if it is at optimal time point based on his/her chronotype.

Therefore, in light of the foregoing discussion, there exists a need to overcome the aforementioned drawbacks associated with the maintenance of the regular activities and a healthy lifestyle thereof.

### SUMMARY

The present disclosure seeks to provide a system for improving general performance of a user. The present disclosure also seeks to provide a method for guiding a user to improve general performance thereof. The present disclosure seeks to provide a solution to the existing problem of inefficient scheduling of daily activities. The present disclosure particularly seeks to provide means fora user to optimise other activities in view of an important activity that takes place at a moment that is not optimal for the user. A further aim of the present disclosure is thus to provide a solution that overcomes at least partially the problems encountered in the prior art and provides an efficient and reliable system for improving general performance of the user. An aim of the present disclosure is also to provide means for determining an optimal time for a given activity, based on the chronotype.

In one aspect, an embodiment of the present disclosure provides a system for guiding a user to improve general performance, the general performance comprising an activity selected from sleep, physical action and cognitive action, the system comprising:

a wearable device to measure at least two parameters associated with the user during an activity period, wherein the parameters are selected from action of the user, temperature of the user and heart rate of the user; and

a computing device operatively coupled to the wearable device, wherein the computing device is operable to:
determine a circadian rhythm of the user based on the measured parameters;
classify the user into a chronotype class based on the determined circadian rhythm;
determine a typical activity schedule for the user based on the measured parameters;
determine an optimal time period in a typical day of the user for at least one of physical action and cognitive action, based on the user's chronotype class;
receive information about an intended activity of the user;
analyse whether the intended activity is within the optimal time period; and
guide the user on carrying out the intended activity, taking into account whether the intended activity is within the optimal time period.

In another aspect, an embodiment of the present disclosure provides a method for guiding a user to improve general performance, the general performance comprising an activity selected from sleep, physical action and cognitive action, the method comprising:
measuring at least two parameters associated with the user during an activity period, wherein the parameters are selected from action of the user, temperature of the user and heart rate of the user;
determining a circadian rhythm of the user based on the measured parameters;
classifying the user into a chronotype class based on the determined circadian rhythm;
determining a typical activity schedule for the user based on the measured parameters;
determining an optimal time period in a typical day of the user for at least one of physical action and cognitive action, based on the user's chronotype class;
receiving information about an intended activity of the user;
analysing whether the intended activity is within the optimal time period; and
guiding the user on carrying out the intended activity, taking into account whether the intended activity is within the optimal time period.

Embodiments of the present disclosure substantially eliminate or at least partially address the aforementioned problems in the prior art and enables the user to improve the performance by acknowledging the circadian rhythm and scheduling the activities accordingly.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 is a schematic illustration of a system for guiding a user to improve general performance, in accordance with an embodiment of the present disclosure;

FIG. 2 is an illustration of steps of a method for guiding a user to improve general performance, in accordance with an embodiment of the present disclosure;

FIG. 3 is an illustration of steps of a method for providing feedback to a user about a circadian rhythm and a chronotype class thereof, in accordance with an embodiment of the present disclosure;

FIG. 4 is an illustration of steps of a method for providing feedback to a user, in accordance with an embodiment of the present disclosure;

FIG. 5 is a schematic illustration of an example of a circadian rhythm of the user and common daily activities associated therewith, in accordance with an embodiment of the present disclosure;

FIG. 6A is an exemplary illustration of measured parameters, including heart rate, activity (action, motion) and temperature depicted as plots to define a circadian rhythm of the user, in accordance with an embodiment of the present disclosure;

FIG. 6B is an exemplary illustration of measured parameters, including heart rate, activity and temperature depicted as lines in a plot to define a circadian rhythm of the user over an extended period of time, in accordance with an embodiment of the present disclosure;

FIG. 6C is an illustration of data point for one line presented and plotted in FIG. 6B, in accordance with an embodiment of the present disclosure;

FIG. 6D is an illustration of an exemplary statistical method to analyse parameters of measured parameters and circadian rhythm, in accordance with an embodiment of the present disclosure;

FIGS. 7A and 7B are illustrations of plots depicting various activity periods for two persons with different chronotype classes over a course of a week, in accordance with an embodiment of the present disclosure;

FIGS. 8A and 8B are illustrations of plots depicting typical hormone levels for persons with two different chronotype classes, in accordance with an embodiment of the present disclosure;

FIGS. 9A and 9B are illustrations of plots depicting optimal times for intended activities for persons with different chronotype classes, in accordance with an embodiment of the present disclosure;

FIG. 10 is an exemplary table for defining chronotype class, in accordance with an embodiment of the present disclosure; and

FIGS. 11A and 11B are illustrations of exemplary user interfaces to be shown to the user for depicting data related to guidance for improving performance of a user, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a system for guiding a user to improve general performance, the general performance comprising an activity selected from sleep, physical action and cognitive action, the system comprising:
a wearable device to measure at least two parameters associated with the user during an activity period, wherein the parameters are selected from action of the user, temperature of the user and heart rate of the user; and
a computing device operatively coupled to the wearable device, wherein the computing device is operable to:
   determine a circadian rhythm of the user based on the measured parameters;
   classify the user into a chronotype class based on the determined circadian rhythm;
   determine a typical activity schedule for the user based on the measured parameters;
   determine an optimal time period in a typical day of the user for at least one of physical action and cognitive action, based on the user's chronotype class;
   receive information about an intended activity of the user;
   analyse whether the intended activity is within the optimal time period; and
   guide the user on carrying out the intended activity, taking into account whether the intended activity is within the optimal time period.

In another aspect, an embodiment of the present disclosure provides a method for guiding a user to improve general performance, the general performance comprising an activity selected from sleep, physical action and cognitive action, the method comprising:
measuring at least two parameters associated with the user during an activity period, wherein the parameters are selected from action of the user, temperature of the user and heart rate of the user;
determining a circadian rhythm of the user based on the measured parameters;
classifying the user into a chronotype class based on the determined circadian rhythm;
determining a typical activity schedule for the user based on the measured parameters;
determining an optimal time period in a typical day of the user for at least one of physical action and cognitive action, based on the user's chronotype class;
receiving information about an intended activity of the user;
analysing whether the intended activity is within the optimal time period; and
guiding the user on carrying out the intended activity, taking into account whether the intended activity is within the optimal time period.

The present disclosure provides the system and method for guiding a user to improve general performance. Furthermore, the present disclosure provides a system for measuring parameters of the user. Based on measured parameters, a circadian rhythm of the user is determined. Furthermore, based on the circadian rhythm, a chronotype class of the user is determined. Subsequently, an intended activity associated with the user is defined. Furthermore, an optimal time period for the intended activity is determined. Beneficially, the present system guides the user to tune, fine-tune or reschedule the daily schedule and to accommodate the intended activity therein. As an example, the present system and method make it possible to help the user, for example a morning person, to prepare for a meeting late in the evening, or alternatively to help an evening person for a physical activity to be carried out during the morning. For example, if the target activity is an activity A1 (for example a meeting) at time Z, the system would give guidance for the user that during a time period Z-3 hours, the user should carry out an activity A2 (for example a long walk in the nature, the length and intensity of which could be determined based on the chronotype and/or fitness and/or readiness of the user).

Indeed, while a regular 24-hour rhythm is good for health, there are sometimes needs to deviate from the regular rhythm. The present description aims at providing a method and system for making this deviation and re-adaptation as easy and smooth as possible. Guiding the user on carrying out the intended activity comprises guiding the user on how to prepare for the upcoming intended activity or intended activity period.

Notably, the present disclosure provides a reliable and an efficient system to maintain a healthy and balanced lifestyle. The present method and system also allow the user to optimise her/his activities in view of an important activity that the user cannot schedule according to her/his optimal needs. Thus, for example, if the user has a scheduled important meeting at 22:00 (at which time the user would normally go to bed), the system can give advice and indications to the user on how to modify the daily schedule and re-schedule other activities accordingly, in order to be in optimal shape for the meeting at 22:00. This is achieved by analysing whether the intended activity is within the optimal time period and guiding the user on carrying out the intended activity, taking into account whether the intended activity is within the optimal time period. Should the intended activity be within the optimal time period, it may not be necessary to give the user any guidance. However, should the intended activity be outside the optimal time period, the system may guide the user, for example to shift physical action to a moment of time where the positive effect of the physical action on mental performance falls within the time of the intended activity.

Throughout the present disclosure, the term “parameter” used herein relates to one of characteristic attributes associated with the user. For example, the parameters include action of the user, temperature of the user, blood pressure of the user, heart rate of the user and so forth. Additionally, the parameters associated with the user provide a basis for monitoring and evaluating the habits of the user. Further, the term “general performance” comprises performance of the user related to activities selected from sleep, physical action and cognitive action. It will be appreciated that the term “activity” relates to actions, movements, and happenings that are either influenced by the user or occur naturally and the term “action” is used when specifying which kind of activity is intended, i.e. a physical action is equal to a physical activity and a cognitive action is equal to a cognitive activity. The term action is however used for clarity purposes. Furthermore, the activity is either performed or observed by the user. Moreover, the activities such as sleep relates to go-to-bed time, wake-up time, quality of sleep (deep or light), quantity of sleep (short or long) and so forth. Additionally, the physical action relates to actions and reactions performed by human body, such as for example exercise, walk, yoga, and the like. The cognitive action relates to work functions that are performed in relation with mind of the user such as for example decision-making, problem-solving, memorising, paying attention, judging and the like. Furthermore, duration for the activity may vary from minutes to couple of hours to couple of months depending upon the nature and characteristics thereof, or even longer. For example, an activity involving a game may last for 30 minutes to 10 hours, depending on the type of the game.

Throughout the present disclosure, the term “wearable device” used herein relates to a device that detects (and possibly responds to) signals, stimuli or to changes in quantitative and/or qualitative features of a given system, or the environment in general and provides a corresponding output. The output is generally a signal that can be converted to human-readable form, for example on a display at the sensor location or transmitted electronically over a network for reading or further processing. The wearable devices may include electronic devices, for example made of a mouldable ceramic material and having a cavity with electronic parts arranged therein. In an example, the wearable device may include a ring, a bracelet, a necklace, a wrist band and the like, that can be easily worn by the user. In another example, the wearable devices may be arranged to be used by or placed in proximity of the user, such as a pillow, a mattress, a chair etc. Further, the wearable devices may be operable to detect the temperature (body temperature, surrounding temperature and the like), motion, direction and the like. Additionally, in some examples, the wearable devices are operable to provide visual, auditory, tactile (e.g., touch, pressure), olfactory (e.g., smell), balance or any combination of perceptual information.

The wearable device is configured to measure at least two parameters associated with the user during an activity period. The parameters are selected from action of the user, temperature of the user and heart rate of the user. In an example, action of the user may include sleep timings (quantity and quality of sleep), eating habits (quantity, quality, time schedule, type of diet), physical actions (exercise, walk, jogging, skiing and so forth), and the like. Furthermore, the wearable device can measure actions, reactions, happenings and so forth that occur in the proximity of the user. In an example, the wearable device is operable to monitor the action of the user, temperature of the user and heart rate of the user. In an instance, the wearable device may monitor the sleeping habits, for example time to go-to-bed, wake-up time, quality of sleep (deep or light) and so forth. Additionally, the wearable device may analyse the duration of good sleep (deep sleep), time between go-to-bed time and falling-asleep time, sleep phases (number of times awaken between midnight or mid of sleep), and so forth. In one example, the wearable device measures inner and outer body temperature of the user. In another example, the wearable device is operable to measure different parameters corresponding to the user, such as blood volume pulse to determine a heart rate of the user. In yet another example, the heart rate is determined by measuring a photoplethysmogram (PPG) from the blood volume pulse. In such cases, a PPG measuring device may be arranged in the wearable device.

One possible action measured may also be motion of the user. By motion in the present context it is understood the movement of the user, which may be measured by any suitable method or apparatus, for example with an accelerometer. The motion may be either actual motion or lack of motion, wherein a lack of motion would typically indicate that the user is resting or sleeping. The measurement of the motion will also indicate the type of movement, for example running and walking give different measurement data. Typically, the wearable device comprises an accelerometer for measuring the motion of the user.

Throughout the present disclosure, the term “computing device” relates to a device that monitors equipment, machines, instruments, and so forth that are attached, coupled or connected therewith. The computing device is operable to receive the information provided from connected equipment and direct the instructions thereto. Optionally, the computing device includes one of an electronic device, controller, electrical or mechanical components and so forth. According to an embodiment, the system comprises the computing device operatively coupled to the wearable device. In one example, the wearable device is coupled to the computing device either through wire or wirelessly. Furthermore, the computing device receives the information about the measured parameters associated with the user through the wearable device. In one example, the computing device may communicate through a user interface, such as a personal digital assistant (PDA), a smartphone, a tablet, a laptop and the like. In particular, the computing device may communicate with the user through a computer application installed in the user interface. Indeed, the computing device can be physically located in the wearable device, in a mobile communication device of the user or at a server.

The computing device is operable to determine a circadian rhythm of the user based on the measured parameters. It will be appreciated that the term “circadian rhythm” used herein relates to a 24-hour cycle of human beings. Furthermore, the circadian rhythm is associated with the regular activities, eating and sleeping habits, and so forth of human beings. Moreover, the circadian rhythm resembles a pattern of activities performed by the human being. Additionally, the pattern of activities performed by the human being repeats in a specified period of time (for example couple of days, a week or a couple of weeks). Based on the measured parameters, the circadian rhythm is determined such as sleeping hours out of 24-hours, sleep and wakeup time, eating and hygiene routine and so forth. Furthermore, the computing device provides a specific pattern of parameters that is being followed by the user for a defined period of time.

Further, the computing device is operable to classify the user into a chronotype class based on the determined circadian rhythm. It will be appreciated that the term “chronotype class” used herein relates to the circadian rhythm of the user with respect to the 24-hour rhythm. The chronotype class is defined based on the concept that each individual has a different circadian rhythm such as difference in sleep timings, eating timings and so forth. That is, each individual has a variation in the alignment of the day and night rhythm, i.e. circadian rhythm to the 24-hour rhythm. Additionally, human beings' internal clocks are not identical to the 24-hour rhythm. Thus, there are variations in sleep timings, eating timings and so forth. Based on the different circadian rhythms, the individuals are classified into various chronotype classes, namely between “Class 1” to “Class 9”, as discussed later. Herein, a person may be classified with chronotype as “Class 1”, if the person follows early sleep time and has low sleep drive; and to the other extreme, a person may be classified with chronotype as “Class 9” if the person follows late sleep time and has high sleep drive. In some examples, the user may be classified into a chronotype class based on other information related to lifestyle and general habits of the user and the like.

The computing device may be operable to define a chronotype class of the user using one or more of criterions of sleep drive comprising low sleep drive, medium sleep drive and high sleep drive, and sleep time comprising early sleep time, intermediate sleep time and late sleep time. That is, the chronotype classification typically includes at least one criterion of sleep drive comprising low sleep drive, medium sleep drive and high sleep drive; and sleep time comprising early sleep time, intermediate sleep time and late sleep time. In case of sleep drive, the classification is defined based on low sleep drive such as individuals used to a short period of sleep (for example 3 to 5 hours or less), medium sleep drive such as individuals used to a moderate period of sleep (for example 5 to 7 hours), and high sleep drive such as individuals used to have a deep and long sleep (for example 7 to 10 hours or more). Similarly, in case of sleep time, the classification is defined based on early sleep time such as individuals used to go to bed early (for example between 7:00 pm and 9:00 pm), intermediate sleep time such as individuals used to go to bed at a well-defined time (for example between 9:00 pm and 11:00 pm), and late sleep time such as individuals used to go to bed late (for example between 11:00 pm and 4:00 am). Therefore, based on observed sleep timings, including the sleep drive and the sleep time, the user can be classified into one chronotype class.

In one embodiment, determination of the circadian rhythm and chronotype classification of the user is carried out after a fixed interval of time. That is, it may not be necessary to determine the circadian rhythm and chronotype classification of the user every time the computing device needs to schedule some activity. In an instance, the circadian rhythm and the chronotype class may be determined after every couple of days or weeks or months, and, in the meantime, the last determined circadian rhythm and the chronotype class is used for all calculation purposes. In some examples, the information about the determined circadian rhythm and chronotype classification is stored and further compared with the past readings to draw conclusions related to health of the user. Indeed, the circadian rhythm and/or chronotype classification can be determined once, when the user starts to use the system, and can be regularly or irregularly updated or checked or adjusted later on.

The computing device is operable to determine a typical activity schedule for the user based on the measured parameters. It will be appreciated that the typical activity schedule relates to a set of regular activities that are performed by the user on a daily basis and in a defined period of time. Furthermore, the typical activity may include go-to-bed time, wakeup time, exercise time, work time, day and night activities and so forth. Based on the measured parameters, the computing device analyses an average time period for a particular typical activity. For example, if the user wakes up between 6 am to 8 am in the morning, then the average time period for the wakeup time would be set at 7 am in the morning. Further, in an example, if the workout timing is 2 to 4 hours daily, then the average time period for workout timing would be 3 hours. Therefore, based on the measured parameters, the typical activity schedule for each of the typical activity is assigned at a defined period of time. The computing device may determine a typical activity schedule for the user based on the statistical or other data. The statistical and other data can be detected or measured from other users or test persons.

According to an embodiment, the computing device is operable to determine an optimal time period in a typical day of the user for sleep and at least one of physical action and cognitive action, based on the user's chronotype class. Furthermore, the typical activity schedule includes a specified time period for each of the typical activity in a typical day of the user such as for example 7 am (wakeup time). In some instances, the typical activities could not be organised in an appropriate way due to irregular time schedule of the user; and additionally, the circadian rhythm of the user does not allow or accommodate the typical activities to be performed at an appropriate time period. In such instances, the computing device helps to determine an optimal time period in a typical day of the user for sleep and at least one of physical action and cognitive action, based on the user's chronotype class. For example, the sleep period may be organised to have a complete 5 to 9 hours of sleep, and thus the go-to-bed timing may be set at 11 pm in night and wakeup time at 5 am in morning. In one example, if the user works at irregular office timings due to late entries at office, thus the go-to-office time may be optimised at 9 am sharp in the typical activity schedule. In another example, the user may be experiencing stress due to continuous working hours in a day; in such a case, an optimised break or recess can be accommodated in between the regular working hours.

The computing device can be operable to generate a schedule for the user based on the measured parameters, wherein the schedule defines time periods for the user to perform one or more various typical activities associated with a day of the user. Furthermore, the measured parameters represent various characteristics of the user such as sleep timings, heart rate, body temperature, day and night actions and so forth. Moreover, the monitored activity parameters are analysed and an appropriate schedule is generated by the computing device. In an instance, the schedule may include various typical activities associated with a day of the user such as meetings, assignments, typical day and night activities and so forth. The computing device formulates the schedule defining time periods for the user to perform the one or more various typical activities associated with a day of the user. For example, the schedule may include various time periods defined for various activities such as wake-up time at 6 am in the morning, exercise till 7 am, 7 am to 9 am for breakfast, lunch at 12 pm, dinner at 8 pm, go to sleep at 10 pm, and so forth. In one example, if the schedule for a day of the user includes a visit to a doctor, attending a meeting, and so forth, the schedule may define the optimal time periods for visiting the doctor, attending the meeting and so forth. Alternatively, the system may give the user guidance on how to amend other activities of the day or what type of activity or meal at which time of the day is desirable in order to perform the intended activity (such as visiting a doctor or attending a meeting).

In one embodiment, the determination of a typical activity schedule for the user is further based on parameters measured during at least two preceding 24-hour periods. Alternatively, the determination may be based on parameters measured during at least two preceding activities. That is, the computing device acquires the information about the time period of the at least two preceding activities and schedules a time period of the typical activity thereafter. Specifically, the computing device may regularise the typical activity schedule based on the priority importance and types of the preceding activities.

Throughout the present disclosure, the term “intended activity” relates to a specific task that is to be performed by the user in a specified time period. Optionally, intended activity may include one of a specific exercise schedule, appointment, healthy eating habit (proper meal timings), proper sleep timing, and so forth. Additionally, intended activity may be the task that the user may be lagging to achieve in the specified time slot, for example to wake up early in the morning, completing an exercise routine, eating the meals of the day at appropriate timings and so forth. Furthermore, in some examples the computing device is operable to suggest at least one of physical training action and cognitive task action to the user based on the circadian rhythm. For example, the suggested intended activity may be a mind muscle coordination, yoga, power exercises, and so forth. The intended activity may also be an important work meeting or any other activity which timing the user cannot freely define, but which is rather imposed to the user. The system is then operable to guide the user on how to achieve best performance at the intended activity. The system may also take into account that a meeting scheduled to take place at 16:00 at the user's regular time zone will take place at 22:00 because the user has travelled to another time zone. The system then helps the user to optimize his/her actions to be in best condition for this task.

Furthermore, the computing device is operable to receive information about an intended activity of the user. In one embodiment, the information about the intended activity is received directly from the user. That is, the user may provide information about the intended activity directly to the computing device, such as scheduling the meal timings at user-specified time periods, regularising the go-to-bed timings and wakeup timings, an important meeting, an upcoming flight (with change of time zone) and so forth. In one embodiment, the information about the intended activity can be received and collected from an electrical calendar of the user such as Google calendar or the information can be collected from emails or messages or Facebook postings using well known computer and artificial intelligence methods. Moreover, the user may provide information about intention of the standard health schedule to be inculcated in the daily schedule thereof. In one example, the user may provide information about an important meeting to be considered as an intended activity, which shall be incorporated with the regular activities in the schedule. In some examples, the computing device receives information about the intended activity that would help the user in regularising the prominent activities for a balanced health and lifestyle.

In one embodiment, the intended activity is defined based on the schedule of the user. In particular, the intended activity is defined in accordance to a regular schedule of the user.

In another embodiment, the intended activity and its timing is given, and the other activities in the schedule of the user are re-scheduled accordingly, in order for the user to achieve optimum performance at the intended activity.

Furthermore, the computing device is operable to analyse whether the intended activity is within the optimal time period. That is, in order to incorporate the intended activity in between the regular activities, an optimised time period for performing the intended activity is to be defined. Additionally, the time period for the intended activity should not collide with any of the time periods of the regular activities. Further, determining the optimal time period for performing the intended activity should be based on the determined circadian rhythm and a type of the intended activity. For example, if an exercise of 30 minutes is to be incorporated, then preferably the optimal time period should be defined in the morning (between wakeup time and breakfast) or in the evening (before dinner). Specifically, if the user wakes up 6 am in the morning, then the exercise can be scheduled around 6:30 am to 7 am. In one example, if an important meeting (on a particular date) is to be scheduled, then the optimal time period for the meeting should be defined within the regular work schedule. The computing device is operable to determine the optimal time period for the intended activity according to the circadian rhythm (wakeup time, go-to-bed time, meal timings, and so forth) of the user. It may be understood that if multiple intended activities are to be incorporated within the regular schedule, then multiple time slots would be defined to accommodate the multiple intended activities. In one example, if the user plans to re-schedule a morning exercise, an important meeting, and so forth along with the regular schedule, then the computing device determines suitable time slots for each of the activities to be performed by the user. In such an instance, the computing device may inform the user to prioritise the activities according to the circadian rhythm thereof.

In one embodiment, the computing device is operable to receive information about a preferred time slot for the intended activity from the user. In particular, the computing device is operable to receive information about a preference, an importance and a priority of the intended activity from the user. Based on the received information, the computing device incorporates the preference, the importance and the priority of the intended activity in the generated schedule. Further, in an embodiment the computing device is operable to adjust the time periods in the schedule to reserve the preferred time slot for the intended activity and to guide the user on actions to be carried out to achieve the optimal performance. The computing device is operable to accommodate the intended activity according to the circadian rhythm and the type of intended activity. In an instance, if an intended activity is to be performed in the time slot of the regular activity, then the computing device adjusts the time periods in the schedule to reserve the preferred time period for the intended activity. That is, the computing device is operable to make adjustments in the regular time schedule to reserve the preferred time period for the intended activity. In an instance, if the user wakes up early and goes to bed early, then the computing device makes the adjustments, such that most of the activities (regular activities and intended activity) are scheduled in early day time. In some embodiments, the computing device is operable to make adjustments in the schedule according to the instructions of the user for a particular intended activity. In one example, if the user targets to ride a bicycle for 30 minutes after performing morning exercises but the regular routine follows a bath and a breakfast thereafter; in such an instance, the computing device shifts the bath and the breakfast after the cycling for 30 minutes; and subsequently, the other day and night activities are shifted 30 minutes henceforward. In another example, if the user wishes to attend a certain meeting at 22 pm next day (which meeting is not in his/her typical schedule and not within in his/her optimal time period), the computing device make adjustments in the next day schedule to reserve the time slot for the meeting. This re-scheduling is accompanied with guidance to the user on how to amend the daily schedule in practice, for example which time is optimal for breakfast, lunch and dinner, and how to schedule and execute physical training during the day before the meeting. The daily schedule can be amended for the one day or some days prior to the rescheduled meeting or any another activity. The computing device may guide the user to shift the amended meal times in series towards the meeting. For example, the computing device can amend and guide the user two days before the meeting, by shifting all meals 1 hour forward from the normal schedule for the first day and by shifting them 2 hours forward from the normal schedule for the second day and so on, until the meeting day. Furthermore, the computing device can reschedule and guide the user to execute a physical training session to happen for example 3 hours before the meeting on the first day and 2 hours before the meeting on the second day and cancel the normally scheduled physical training session or amend it to be much shorter and lighter for one or more days prior to the meeting.

In one embodiment, the computing device is operable to remind the user about the time slot for performing the intended activity. Beneficially, the computing device is operable to provide an alarm to the user about performing the intended activity. Optionally, the computing device may provide notification about the intended activity. More optionally, the notification may be provided through an external source to remind the user. For example, the computing device may be operable to store contact information about health and fitness specialists in a database. In such an instance, on receiving a request from the user for a health guide, the computing device may check the database and provide an appropriate information for the relevant contact and further, schedule a meeting with such contact. Furthermore, the health and fitness specialists may advice the user on a regular basis, referring to health information based on the circadian rhythm of the user. In one example, the computing device may remind the user through one of the user interfaces such as PDA, smartphone, tablet, laptop, and the like.

In one embodiment, the computing device is operable to detect one or more acts of a user related to performing the intended activity and provide feedback to the user based on the one or more acts. That is, the computing device receives information about the execution and time period of the intended activity. Moreover, the computing device monitors the one or more acts performed by the user related to execution of the intended activity. Thereafter, the computing device provides feedback to the user based on the received information about execution and time period of the intended activity. In this context, by “acts” are meant various actions that form part of the activity, such as preparation for the activity itself. For example, if an intended activity such as a target exercise is scheduled to be performed at a specified time slot, the computing device is operable to receive information about the time duration, quality, scheduled timing, and so forth of the target exercise. Furthermore, the computing device provides a feedback on performance of the user while carrying out the target exercise. The feedback may include possible improvements that could be made by the user to improve the experience of the exercise and/or health benefits attained by performing the exercise.

According to an embodiment, the computing device is operable to guide the user on carrying out the intended activity, taking into account whether the intended activity is within the optimal time period. That is, the computing device receives information about the intended activity and the optimal time period for execution thereof. Subsequently, the computing device verifies the availability of an optimal time period for the execution of the intended activity. Furthermore, the computing device advices the user to shift the time slot of the activities to accommodate the intended activity in a preferred time slot. In an example, if the user loses a healthy diet schedule due to irregularity of the sleep timings, day and night activities, meal timing, external addictions (smoking, consumption of alcohol and junk food) and the like, the computing device may guide the user to take measures for recovering the health by following the prescribed schedule provided thereto.

In one embodiment, the computing device is operable to incorporate time zone changes into the schedule of the user, as mentioned above. In some cases, the user may frequently travel to different time zones for various purposes. In such cases, the computing device is operable to guide the user on scheduling the typical activities and also the intended activities as per the real time zone of the current location of the user. It may be understood that the user may experience various changes in the daily activity schedule due to change in time zone. However, the computing device makes the shift to new time zone in such a manner that there are no collisions between the scheduled activities (either typical activity or intended activity).

In some embodiments, the computing device is operable to reschedule at least one activity to improve performance of the intended activity. Further, the at least one activity to be rescheduled is selected from one or more of the meal timings, waking-up time, going-to-bed time, reading time or some other typical activity. In one example, if a 5-kilometre walk (intended activity) is to be accommodated in the daily activity schedule, then the computing device may push the regular breakfast time within optimal time period therefor. In such an instance, the computing device may further reschedule the sleep timing (like scheduling earlier go-to-bed time) to improve health benefits from the walk. In another example, if 1-hour of swimming is selected as an intended activity on a regular basis, the computing device may guide the user to take light meals, go early to bed and wakeup early. Additionally, the computing device may inform the user about the benefits of including such activities to improve the performance.

### DETAILED DESCRIPTION OF THE DRAWINGS

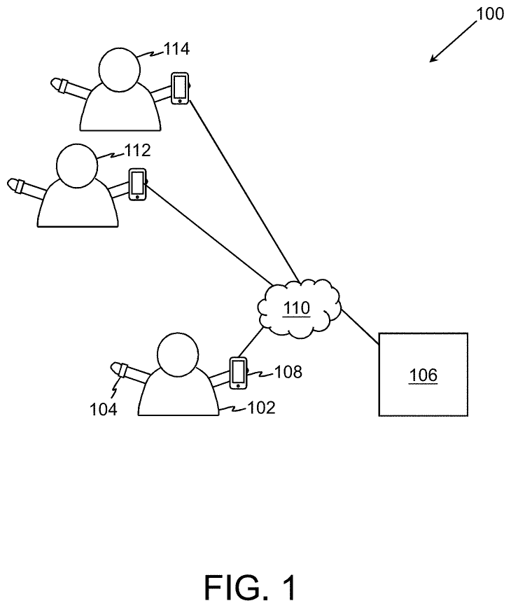

Referring to FIG. 1, there is shown a schematic illustration of a system 100 for guiding a user 102 to improve general performance of the user 102, in accordance with an embodiment of the present disclosure. The system 100 comprises a wearable device 104 arranged to be worn by the user 102 and configured to measure parameters associated with the user 102. The system 100 also comprises a computing device 106 operable to determine a circadian rhythm of the user based on the measured parameters and classify the user into a chronotype class based on the determined circadian rhythm. The computing device 106 is further operable to determine a typical activity schedule for the user based on the measured parameters, determine an optimal time period in a typical day of the user for sleep and at least one of physical action and cognitive action based on the user's chronotype class, receive information about an intended activity of the user, analyse whether the intended activity is within the optimal time period, guide the user on carrying out the intended activity taking into account whether the intended activity is within the optimal time period, and the like. Optionally, the system 100 further comprises a mobile device 108 to receive data related to measured parameters from the wearable device 104 and a network 110 for allowing communication between the mobile device 108 and the computing device 106 for sending and receiving information. More optionally, the system 100 may be configured to allow the computing device 106 to communicate with multiple users, including users 112 and 114 in addition to the user 102, such that the computing device 106 can carry out above listed functions for the users 112 and 114 as well.

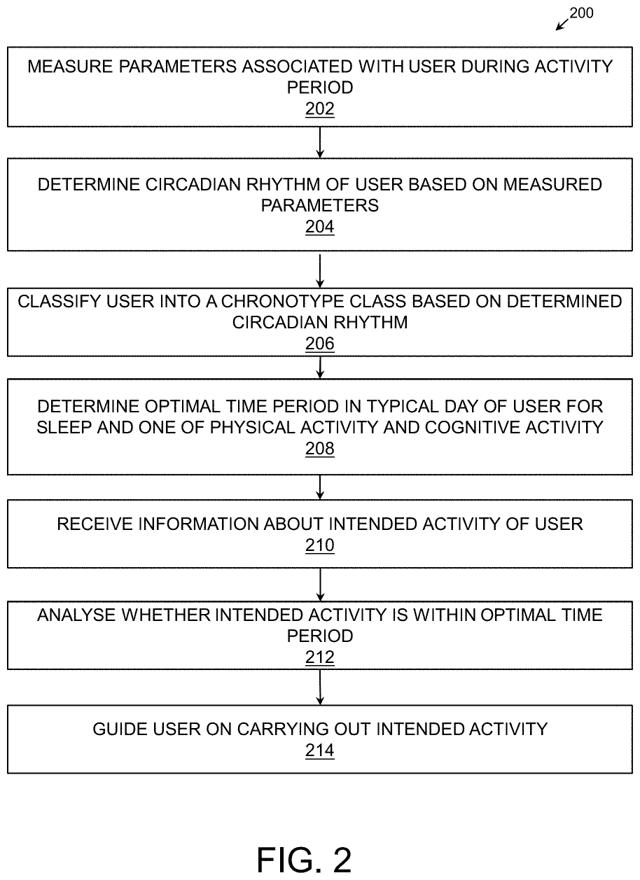

Referring to FIG. 2, there is shown an illustration of steps of a method 200 for improving performance of a user, in accordance with an embodiment of the present disclosure. At step 202, at least two parameters associated with the user are measured during an activity period. At step 204, a circadian rhythm of the user is determined based on the measured parameters. At step 206, the user is classified into a chronotype class based on the determined circadian rhythm. At step 208, an optimal time period is determined in a typical day of the user for sleep for at least one of physical action and cognitive action. At step 210, information about an intended activity of the user is received. At step 212, the intended activity is analysed for whether it is within the optimal time period. At step 214, the user is guided on carrying out the intended activity, taking into account whether the intended activity is within the optimal time period.

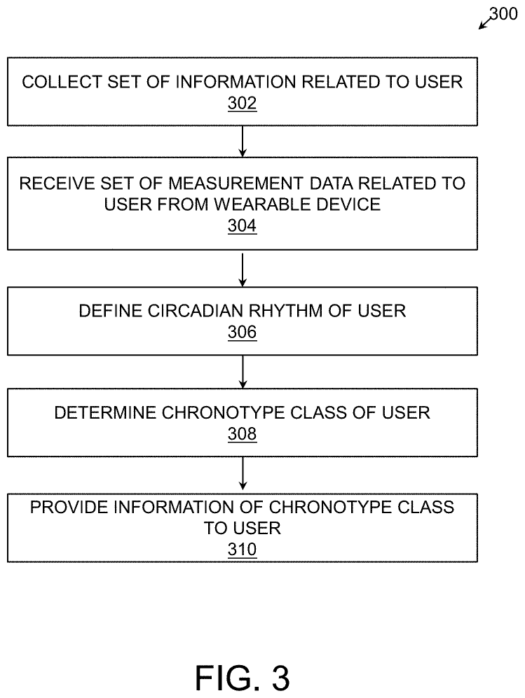

Referring to FIG. 3, there is shown an illustration of steps of a method 300 for providing feedback to a user about a circadian rhythm and a chronotype class thereof, in accordance with an embodiment of the present disclosure. At step 302, the set of information related to the user is collected. At step 304, the set of measurement data related to user is received from the wearable device. At step 306, a circadian rhythm of the user is defined. At step 308, a chronotype class of the user is determined. At step 310, the information about the chronotype class is provided to the user

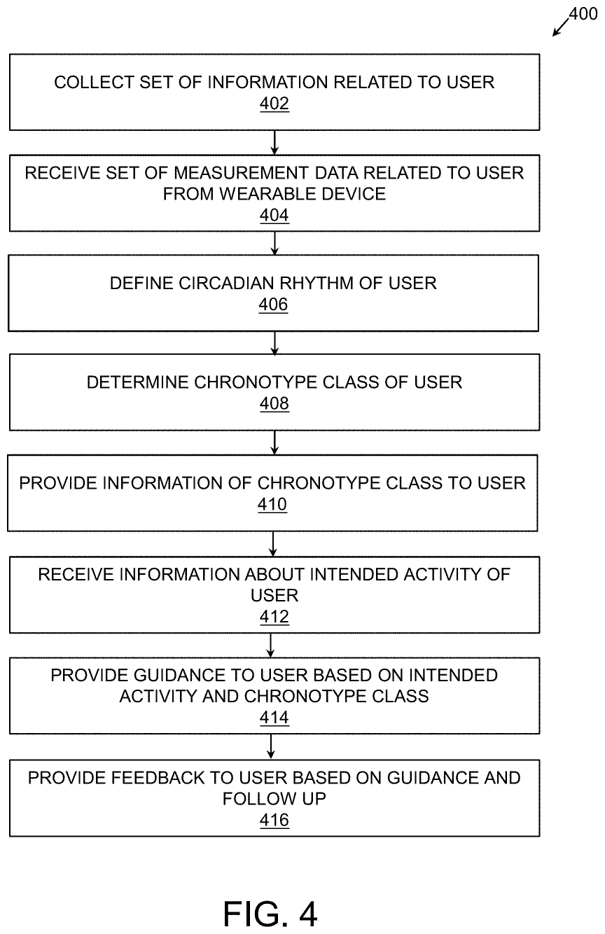

Referring to FIG. 4, there is shown an illustration of steps of a method 400 for providing feedback to a user, in accordance with an embodiment of the present disclosure. At step 402, the set of information related the user is collected. At step 404, the set of measurement data related to user is received from the wearable device. At step 406, a circadian rhythm of the user is defined. At step 408, a chronotype class of the user is determined. At step 410, information about the chronotype class is provided to the user. At step 412, information about the intended activity of the user is received. At step 414, guidance is provided to the user based on the intended activity and chronotype class in order to reach the intended activity. At step 416, feedback is provided to user based on the guidance, and further follow up is performed, as necessary.

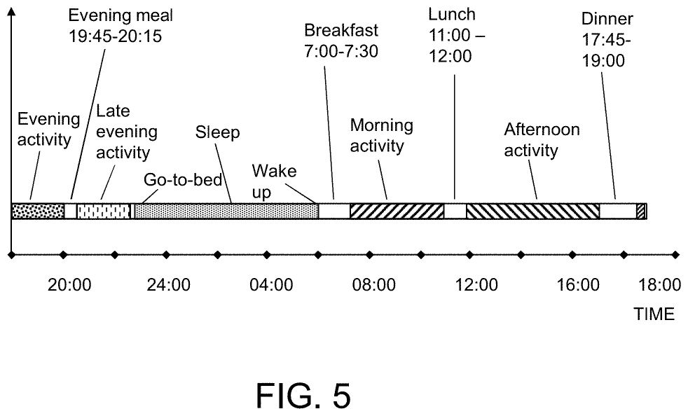

Referring to FIG. 5, there is shown a schematic illustration of an example of a circadian rhythm of the user and common daily activities associated therewith, in accordance with an embodiment of the present disclosure. Herein, the circadian rhythm represents the activity schedule of the user and time duration with respect to the 24-hour clock. As shown, based on the circadian rhythm, the daily schedule includes day and night activities, meals (breakfast, lunch, dinner), and so forth. It may be understood that the time duration for various activities may vary depending upon chronotype, occupation, geographical location of the user.

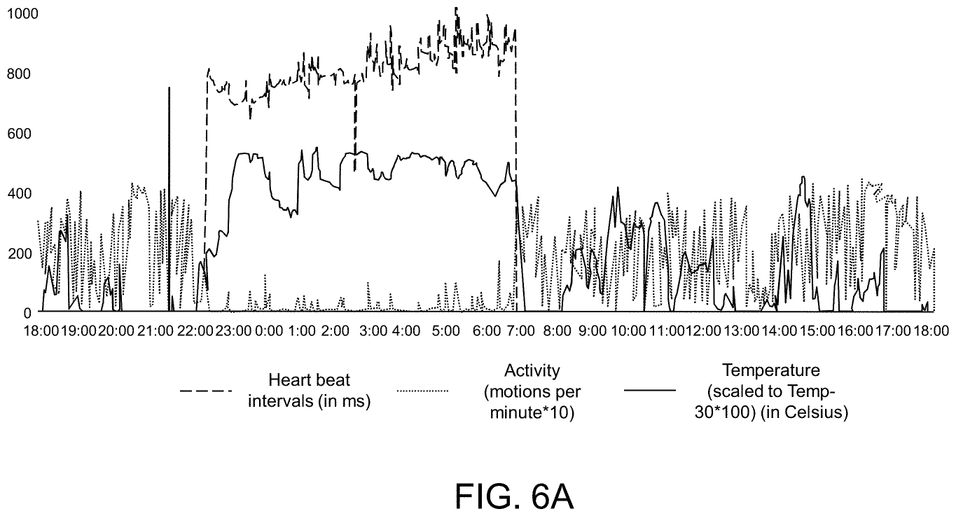

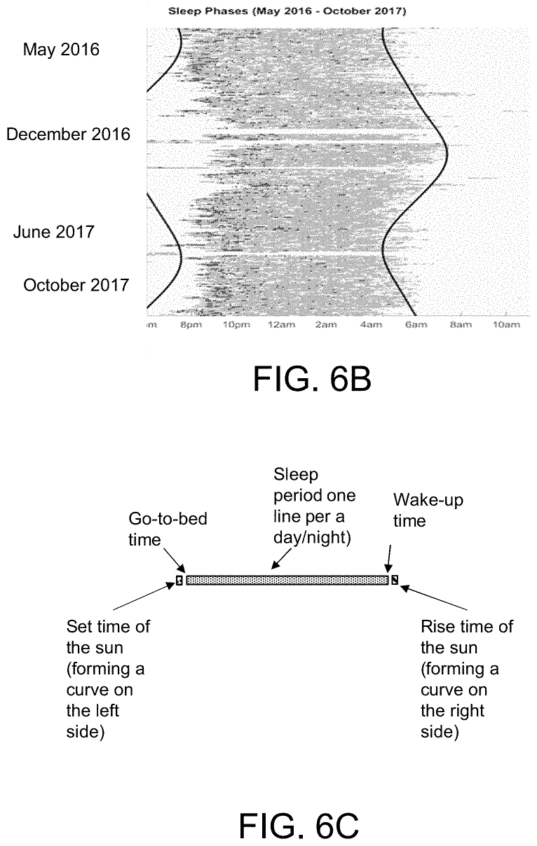

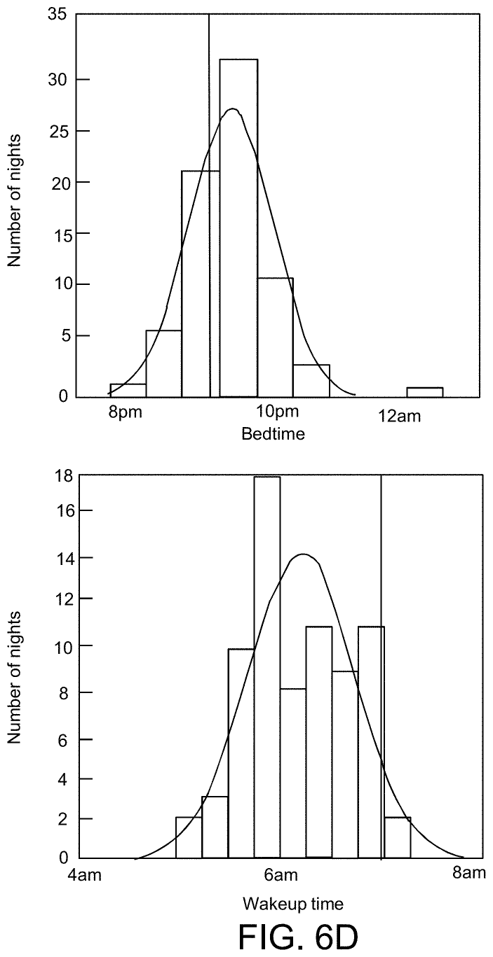

Referring to FIG. 6A, there is shown an illustration of measured parameters, including heart rate, action and temperature depicted as plots to define a circadian rhythm of the user, in accordance with an embodiment of the present disclosure. In this case, the action is based on motion sensor-data, measured with a 3-axis accelerometer. As shown, the graph traces variation of heart rate, activity and temperature with respect to 24-hour clock. Herein, the measured parameters represent the phases of circadian rhythm. As may be seen, skin temperature is higher at night, and the activity, in terms of motions, is lower at night.

Referring to FIG. 6B, there is shown an illustration an illustration of circadian rhythm variation during a long time period (about 18 months), in accordance with an embodiment of the present disclosure. Each horizontal line/bar represents the sleep phase of each day (circadian rhythm) namely go-to-bed time, sleep period and wake-up-time. The go-to-bed time, sleep period and wake-up-time are defined from measured parameters (including heart rate, action and temperature). As shown, the graph shows variation in circadian rhythm over seasons and especially following sun rise and set times. These seasonal and other variations can be taken into consideration in the system. Furthermore, the graph also depicts variation in sleep time with respect to days and months. Referring to FIG. 6C, there is shown an illustration of data point for one line presented and plotted in FIG. 6B, in accordance with an embodiment of the present disclosure. As shown, the line shows the sleep time with respect to rising and setting of sun for one day.

Referring to FIG. 6D, there is shown an illustration of an exemplary statistical method to analyse parameters of measured parameters and circadian rhythm, in accordance with an embodiment of the present disclosure. As shown, histograms represent the go-to-bed times and wakeup times for a number of days. From the depicted histogram, information about an average sleep time of the user may be derived based on go-to-bed times and wakeup times.

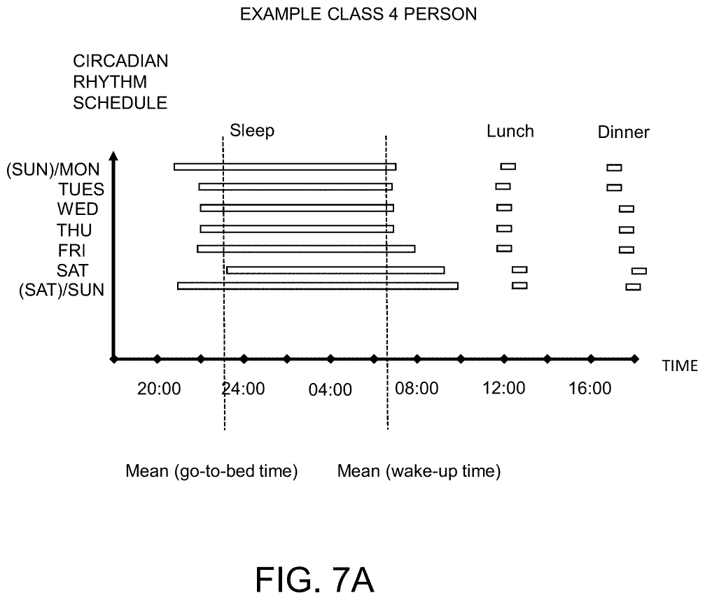

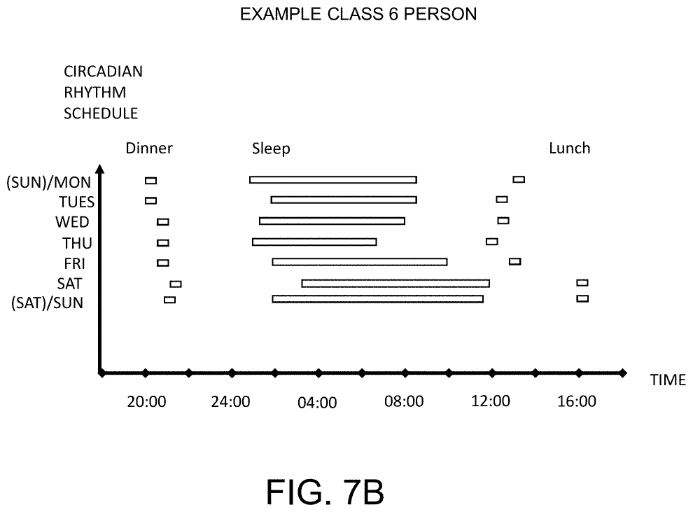

Referring to FIGS. 7A and 7B, there are shown illustrations of plots depicting various activity periods for two persons with different chronotype classes over a course of a week, in accordance with an embodiment of the present disclosure. FIG. 7A provides a graph which depicts schedule for a person with one chronotype class that goes to sleep early and manages all the activities earlier in the day time. FIG. 7B provides a graph which depicts schedule for a person with one chronotype class that goes to sleep late and manages all the activities later in the day time.

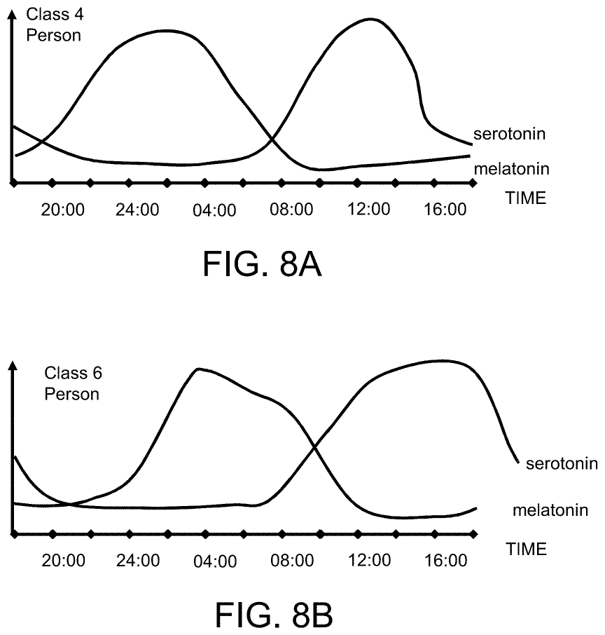

Referring to FIGS. 8A and 8B, there are shown illustrations of plots depicting typical hormone levels for persons with two different chronotype classes, in accordance with an embodiment of the present disclosure. As shown, FIG. 8A provides a graph which depicts hormone levels for a person with “Class 4” as the chronotype class that goes to sleep early. It may be seen that for such person, the serotonin secreted in the morning time peaking at 1 PM, and melatonin secreted in the evening, and peaking at 1 AM. However, the FIG. 8B provides a graph which depicts hormone levels for a person with “Class 6” as the chronotype class that goes to sleep late. It may be seen that for such person, the serotonin and melatonin hormones are secreted a bit late than the person in class 4. Indeed, serotonin is secreted in the morning time peaking at 4 PM, and melatonin secreted in the late night or early morning and peaking at 4 AM.

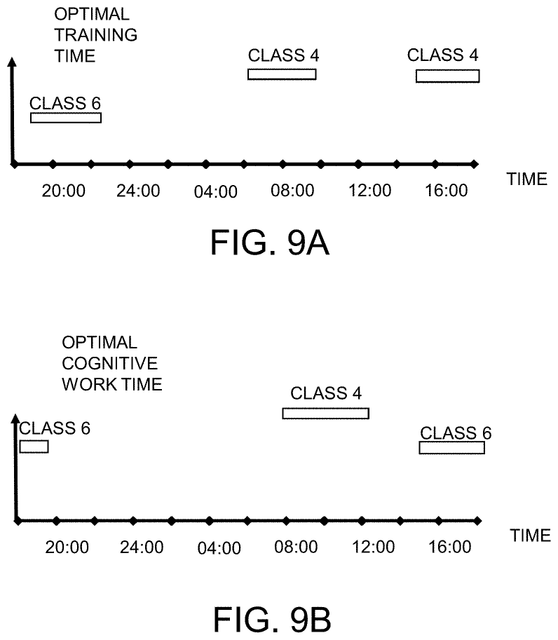

Referring to FIGS. 9A and 9B, there are shown illustrations depicting optimal times for intended activities for persons with different chronotype classes, in accordance with an embodiment of the present disclosure. As shown, bars herein depict the training and cognitive work activities related to persons having different chronotype classes. It may be understood that based on the time schedule of the training and cognitive work activities, optimal times for intended activities can be obtained.

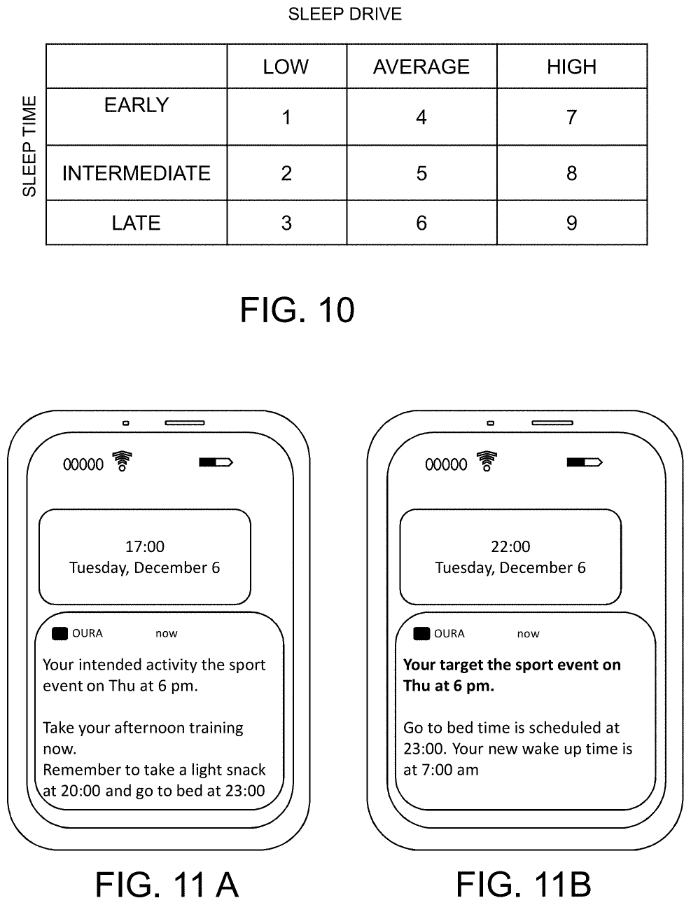

Referring to FIG. 10, there is shown an exemplary table for defining chronotype class, in accordance with an embodiment of the present disclosure. As shown, the table defines the chronotype classes based on sleep time and sleep drives. As discussed in the above paragraphs, defined chronotype classes may be utilized for defining schedule for execution of intended activities within optimal time periods.

Referring to FIGS. 11A and 11B, there are shown illustrations of exemplary user devices with user interfaces for displaying data related to guidance for improving performance of a user, in accordance with an embodiment of the present disclosure. As shown, the user interfaces may receive information about multiple activity parameters, chronotype classes (based on circadian rhythm), intended activities, time slots, and the like. Furthermore, the user device, via the user interface, may guide the user to achieve the intended activities based on the collected information and computation carried in the computing device. Additionally, the user interfaces may provide scheduled notification to the user about the training activities, meal timings, sleeping timings, and the like.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for guiding a user to improve general performance, the general performance comprising an activity selected from sleep, physical action and cognitive action, the system comprising:
a wearable device to measure at least two parameters associated with the user during an activity period, wherein the parameters are selected from action of the user, temperature of the user and heart rate of the user; and
a computing device operatively coupled to the wearable device, wherein the computing device is operable to:
determine a circadian rhythm of the user based on the measured parameters;
classify the user into a chronotype class based on the determined circadian rhythm;
determine a typical activity schedule for the user based on the measured parameters;
determine an optimal time period in a typical day of the user for at least one of physical action and cognitive action, based on the user's chronotype class;
receive information about an intended activity of the user;
analyse whether the intended activity is within the optimal time period; and
guide the user on carrying out the intended activity, taking into account whether the intended activity is within the optimal time period.

2. A system according to claim 1, wherein the computing device is configured to generate a schedule for the user based on the measured parameters, wherein the schedule defines time periods for the user to perform one or more various typical activities associated with a day of the user.

3. A system according to claim 1, wherein the determination of a typical activity schedule for the user is further based on parameters measured during at least two preceding 24-hour periods.

4. A system according to claim 2, wherein the computing device is configured to adjust the time periods in the schedule to reserve a preferred time slot for the intended activity.

5. A system according to claim 1, wherein the computing device is configured to define a chronotype class of the user using one or more of criterions of:
sleep drive comprising low sleep drive, medium sleep drive and high sleep drive; and
sleep time comprising early sleep time, intermediate sleep time and late sleep time.

6. A system according to claim 1, wherein the information about the intended activity is received directly from the user.

7. A system according to claim 2, wherein the intended activity is defined based on the schedule of the user and the computing device is operable to incorporate time zone changes into the guiding.

8. A system according to claim 1, wherein the computing device (106) is configured to:
detect one or more acts of user related to carrying out of the intended activity; and
provide feedback to the user based on the one or more acts.

9. A system according to claim 1, wherein the computing device is configured to guide the user to reschedule at least one activity to improve performance of the intended activity.

10. A system according to claim 9, wherein the at least one activity to be rescheduled is selected from eating at least one meal, waking up, going to bed and physical action.

11. A system according to claim 1, wherein the computing device is configured to provide reminders to the user by sending a message when the user is not performing the intended activity.

12. A method for guiding a user to improve general performance, the general performance comprising an activity selected from sleep, physical action and cognitive action, the method comprising:
measuring at least two parameters associated with the user during an activity period, wherein the parameters are selected from action of the user, temperature of the user and heart rate of the user;
determining a circadian rhythm of the user based on the measured parameters;
classifying the user into a chronotype class based on the determined circadian rhythm;
determining a typical activity schedule for the user based on the measured parameters;
determining an optimal time period in a typical day of the user for at least one of physical action and cognitive action, based on the user's chronotype class;
receiving information about an intended activity of the user;
analysing whether the intended activity is within the optimal time period; and
guiding the user on carrying out the intended activity, taking into account whether the intended activity is within the optimal time period.

13. A method according to claim 12, further comprising generating a schedule for the user based on the measured parameters, wherein the schedule defines time periods for the user to perform one or more various typical activities associated with a day of the user.

14. A method according to claim 12, further comprising receiving information about a preferred time slot for the intended activity from the user.

15. A method according to claim 14, further comprising adjusting the time periods in the schedule to reserve the preferred time slot for the intended activity.
